(19) United States
(12) Patent Application Publication
(10) Pub. No.: US 2016/0137693 A1
(43) Pub. Date: May 19, 2016

(54) PEPTIDE NUCLEIC ACID PROBE (PNA), KIT AND METHOD FOR DETECTION OF ASPERGILLUS FUMIGATUS AND APPLICATIONS THEREOF

(71) Applicants: UNIVERSIDADE DO MINHO, Braga (PT); UNIVERSIDADE DO PORTO, Porto (PT); BIOMODE - BIOMOLECULAR DETERMINATION, S.A., Braga (PT)

(72) Inventors: Laura Isabel Macieira CERQUEIRA, Braga (PT); Carina Manuela Femandes ALMEIDA, Moure (PT); Nuno Filipe Ribeiro Pinto DE OLIVEIRA AZEVEDO, Braga (PT); Maria Joao Lopes DA COSTA VIEIRA, Braga (PT)

(73) Assignees: UNIVERSIDADE DO MINHO, Braga (PT); UNIVERSIDADE DO PORTO, Porto (PT); CHROMOPERFORMANCE, S.A., Barco-GMR (PT)

(21) Appl. No.: 14/902,375

(22) PCT Filed: Jun. 20, 2014
(86) PCT No.: PCT/PT2014/000042

§ 371 Date: Dec. 31, 2015

(30) Foreign Application Priority Data
Jul. 3, 2013 (PT) 107037

(51) Int. Cl. C07K 7/02; C12Q 1/68

(52) U.S. Cl. CPC C07K 7/02; C12Q 1/6895

(57) ABSTRACT

The present invention refers to the development of a Peptide Nucleic Acid (PNA) probe for the detection and discrimination of Aspergillus fumigatus in different types of samples.

PNA is a synthetic molecule analogue to DNA that, due to its physicochemical properties, allows a faster analysis with higher sensitivity than the DNA probes.

These probes are combined with Fluorescence in situ hybridization (FISH), a molecular biology technique that allows the detection of Aspergillus fumigatus in diverse clinical samples, such as blood, serum, sputum, bronchoalveolar lavage fluid and biopsies.

The combination of these two technologies rendered the FISH procedure faster, simpler and more efficient.

The present invention also includes the development of the kit of detection and respective procedure for the Aspergillus fumigatus identification in clinical samples.

### FIELD OF THE INVENTION

This invention relates to a process for the detection of microorganisms clinically relevant. For that purpose, a PNA probe for the detection and discrimination of Aspergillus fumigatus was developed.

In addition to the probe, the present invention includes the PNA FISH procedure and its application to a kit for the detection of Aspergillus fumigatus in biological samples. Therefore it has clinical application.

### BACKGROUND OF THE INVENTION

Aspergillus fumigatus is a saprophyte filamentous fungus that feeds on decaying organic matter and is able to form a type of spore, named conidium, which can survive in a wide range of aggressive environments. This protection strategy provides ubiquity to Aspergillus fumigatus allowing spreading through the air and colonizing new ecological niches. This fungus can colonize human respiratory mucosa. Depending on the host immune system, the inhaled conidia can lead to disease. In fact, A. fumigatus is the main causative agent of pulmonary infections, namely Invasive Aspergillosis (IA), a condition that is particularly harmful for immunocompromised individuals that need an organ or stem cell transplant, suffer from asthma or tuberculosis, or have an HIV infection.

As this is not the only filamentous fungus that spreads through the air, this species must have some particular pathogenic features.

In fact, virulence can partly be explained by thermotolerance, since A. fumigatus can grow at a broad range of temperatures. It grows well at 37° C. but it can survive at temperatures over 50° C. Additionally, the small diameter of the conidia (2-3 μm) and their peculiar cell wall composition allows them to travel through the respiratory system towards the pulmonary alveoli, where they can deposit. The increased resistance to environmental adverse conditions such as host immune response and oxidative stress represents another characteristic that distinguishes this fungus from others not so frequently harmful.

It is possible to explain IA development through the A. fumigatus infectious life cycle within human host.

After deposition in the pulmonary space, A. fumigatus may start a pathogenic behaviour in vulnerable hosts by epithelial tissue adherence and endocytosis. Within epithelial cells, conidia start swelling and begin to germinate. The germinated hyphae can escape from the epithelial cells, infiltrate blood vessels and induce endothelial cell damage. Subsequently it disseminates through circulation, spreading infection to other organs. The complexity of all the mechanisms involved and the great resistance capacity of A. fumigatus to antifungal substances makes IA hard to cure leading to high mortality rates.

An accurate early diagnosis using clinical samples such as bronchoalveolar lavage fluid (BAL), sputum and blood, among others, is then crucial for treatment success. Until now, diagnosis relies on non-specific techniques, such as direct microscopy visualization and serologic tests (ELISA) targeting the fungi cell wall components galactomannan and (1,3)-β-D glucan, or on fastidious and time consuming culturing methods. PCR based molecular techniques have been applied as a good alternative but lack of methodology standardization and the possibility of undergoing false positive results are the main obstacles for the extended use of this technique.

Fluorescence in situ hybridization (FISH) has been showing promising results since it can be applied directly in samples. This technology detects with very high specificity the microorganisms of interest by targeting oligonucleotide probes to specific ribosomal RNA (rRNA), currently with high copy numbers within cells. More recently it has been developed and optimized peptide nucleic acid (PNA) probes for microorganisms detection. PNA molecules are DNA mimics that have the negatively charged sugar-phosphate backbone replaced by an achiral, neutral polyamide backbone formed by repetitive N-(2-aminoethyl)glycine units.

Although PNA molecules don't have pentoses, a specific hybridization between PNA and nucleic acid complementary sequences, still occur by hydrogen bounds, according to the Watson-Crick rules (U.S. Pat. No. 5,539,082).

The neutral PNA molecule characteristic is responsible for a higher thermal stability (high Tm) between PNA/target sequences (rRNA or DNA double stranded), comparing to the traditionally used DNA probes. Due to this high affinity, PNA probes normally have sequences relatively smaller (13-18 nucleotides) than DNA sequences. Normally DNA probes have at least 18 nucleotides (Lomakin, 1998) due to its poor stability and low melting temperature (Tm), also requiring additional fixation and permeabilization process with enzymes or other agents. Moreover, the PNA molecules present more resistance to nucleases and proteases than DNA molecules.

When PNA probes are attached to a fluorochrome dye, they can be detected by epifluorescence microscopy or flow cytometry.

This technique has provided more prompt and robust results in clinical samples than the traditional culture methods, proving its efficacy, speed, sensitivity and specificity. It has been applied in a wide range of microbiology fields, including pathogenic microorganisms detection in samples of human, food or environmental origin.

Several PNA probes have been developed and optimized for a wide range of microorganisms, including bacteria, Candida species and filamentous fungi (Perry-O'Keefe et al, 2001; Cerqueira, et al, 2011; Almeida et al, 2010; Oliveira et al, 2001; Teertstra et al, 2004; Shinozaky et al, 2010).

It is important to notice that, although very robust after optimized, the development of PNA FISH methods is, just as the development of PCR methods, extremely demanding and requires a great knowledge of the chemical and physical characteristics of the different parameters involved.

Furthermore, it is well known that having a PNA FISH method working for an organism does not warrant that other sequences targeting the same organism will function. Regarding A. fumigatus, a PNA FISH method was not developed until now.

### SUMMARY OF THE INVENTION

The present invention refers to a peptide nucleic acid (PNA) probe for the detection of the Aspergillus fumigatus (that is, identification or quantification).

The probe described in the present invention recognizes the microorganism 28S rRNA or the genomic sequences corresponding to the mentioned rRNA. The PNA probes have physiochemical characteristics that are inherent to its structure and when they are applied to a FISH-based method, allow a faster, more robust and more specific analysis than using a DNA.

One of the advantages of this method is that the probe works robustly in a wide variety of biological samples, which usually does not happen with the other detection molecular methods.

Another relevant aspect is the time required for detection. The method here developed matches the best times reported for the remaining molecular methods, even when the type of sample requires an enrichment step prior to the analysis. The rapidity and the reliability of the method can determine the appropriate and timely treatment of infections for clinical perspectives.

Another aspect of the present invention is related to the development of a kit based on the application of this probe to fluorescence in situ hybridization (FISH), allowing the detection of Aspergillus fumigatus in a broad range of biological samples, in a prompt and simple way.

In a preferred embodiment of the present invention, the PNA probe here described allows the detection of the target sequence in rRNA, in rDNA or in complementary sequences of the rRNA of Aspergillus fumigatus.

On of the embodiments of the present invention is the description of a PNA probe used for the detection and/or quantification of Aspergillus fumigatus characterized in that it has at least 86% of similarity to the sequence SEG ID No. 1-15′-ACA GAG CAG GTG ACA-3′, preferably 87%, 88%, 89%, 90%, 91%, 92%, 93%, 94%, 95%, 96%, 97%, 98%, 99%, 100% of similarity to the sequence SEG ID No. 1-5′-ACA GAG CAG GTG ACA-3′.

In a more preferable embodiment of the present invention, the previously described sequences are linked to at least one type of detectable fraction. The type of detectable fraction to be used may be selected from one of the following groups: a conjugate, a branched detection system, a chromophore, a fluorophore, radioisotope, an enzyme, a hapten or a luminescent compound, among others.

As an example, the fluorophore group can be at least one of the following (not limited to): Alexa series fluorophores, cyanines, 5- (and -6) Carboxy-2′,7′-dichlorofluorescein, 5-ROX (5-carboxy-X-rhodamine, triethylammonium salt), among others.

It is still the subject of the present invention a kit for the detection of the presence or absence and/or quantification of Aspergillus fumigatus in clinical samples.

In a more preferable embodiment of the present invention, the kit may additionally present at least one of the following solutions: a fixation solution, a hybridization solution and a washing solution.

In yet another preferred embodiment of the present invention, the fixation solution can comprise paraformaldehyde and ethanol, namely 2-8% (weight/vol) of paraformaldehyde and 25-90% (vol/vol) of ethanol and/or hybridization solution may comprise formamide.

It is still the object of the present invention the description of a method for the detection of Aspergillus fumigatus or for the detection of Aspergillus fumigatus in biological samples, which uses the PNA probe mentioned earlier and which comprises the following steps:
Contact of the PNA probe with biological samples;
Hybridization of the PNA probe with the target sequence of the microorganisms present within the biological samples;
Detection of the hybridization as an indication of the mentioned detection and quantification in the biological samples, the hybridization may be preferably carried our by fluorescence.

The biological samples can be taken from blood, sputum, bronchoalveolar lavage fluid air, biopsies, air, food, water, among others.

It is still the object of the present invention the use of the PNA probes described earlier, the use of the kit described earlier and the methodology to be applied in a detection of Aspergillus fumigatus, or detection of Aspergillus fumigatus in biological samples.

### GENERAL DESCRIPTION OF THE INVENTION

The present invention comprises the PNA probe, reagents, methods and a kit intended for the detection of Aspergillus fumigatus strains.

The higher specificity of the PNA probes (in relation to DNA probes) allows a better discrimination between related nucleotide sequences with one or two mismatches.

In the present invention this aspect is particularly relevant, since the difference between A. fumigatus strains and some other species is precisely one base for this nucleotide region.

According to this invention, the PNA probe here described allows the detection of the target sequence in the rRNA rDNA, or in complementary sequences in the Aspergillus fumigatus rRNA.

The probe of this invention is used for in situ hybridization analysis of A. fumigatus optionally present in one sample, preferably using the technique of fluorescent in situ hybridization.

The PNA probe described in this invention has 15 nucleotides with the following nucleotidic sequence:

SEQ ID No. 1
5′- ACA GAG CAG GTG ACA- 3′

However, the probe to be used in the detection can present at least 86% of identity to the sequence SEQ ID No. 1.

This probe is applied to the analysis by fluorescence in situ hibridization (FISH).

The development of the PNA FISH probe was carried out in silico using specific software. The selection of the probe sequence was performed by aligning rDNA sequences of the target microorganism. This allowed the identification of potentially useful regions, which will be then evaluated based on other parameters such as specificity, hybridization temperature, percentage of guanine/cytosine, bond free energy and secondary structure.

After the probe design and synthesis, the three steps of FISH procedure, fixation/permeabilization, hybridization and wash, have to be developed and optimized for the selected probe.

This process usually involves the following parameters: temperature, concentration of formamide and ethanol, and hybridization and washing times.

A well-succeeded hybridization afterwards allows inferring about the presence/absence and even the concentration of a microorganism by fluorescence microscopy, flow cytometry or real time PCR. The detected fluorescent signal is generally the result of the specific binding of the small probes to tens or hundreds of rRNA copies present in the fungi cytoplasm. That detectable fraction of the probe, which reports the existence of a stable complex formed by the probe and the target, is selected from one of the following groups: a conjugate, a branched detection system, a chromophore, a fluorophore, radioisotope, an enzyme, a hapten or a luminescent compound.

In the context of the present invention can also be used blocking probes with no detectable fraction, to reduce or eliminate hybridization of the PNA probe to not desirable sequences.

The method described in this invention comprises the contact of a sample with the PNA probe described above. According to the method, microorganisms in a sample are detected, identified or quantified, correlating hybridization of the PNA sequence to the target sequence, performed in suitable hybridization conditions. Consequently, the analysis is based on a single test with a definitive opinion. In contrast, the current routine methods for analysis of microorganisms are based on multiple phenotypic characteristics involving multiple tests.

It is still the object of the present invention a kit suitable for carrying out the test to detect, i.e., to identify or measure the A. fumigatus present in samples. The kit comprises at least one PNA probe and other selected reagents or compounds needed to perform in situ hybridization tests.

Preferably, the method intends to be a diagnostic adjuvant for therapeutic decision. Using a patient sample it is determined the presence of A. fumigatus strains. Thus, this will allow the adequate clinical treatment in accordance with the results obtained.

The PNA probes can be applied directly on the sample prepared on a slide, since the application of these probes doesn't involve the use of reagents or enzymes for the permeabilization of the cellular membranes before the hybridization.

However, some of the compounds that are frequently used in hybridization are required.

Therefore, the probes are normally included in more user-friendly kits.

There are already disclosed examples of kits that use PNA probes for the electrophoretic separation of DNA samples (US2005053944, WO9712995, EP1477572). If the desired approach involves the PNA FISH analysis by flow cytometry, the probe could be applied to the sample in suspension, using the same hybridization compounds.

### DETAILED DESCRIPTION OF THE INVENTION

### I-DEFINITIONS

a) As used herein, the term “nucleotide” includes natural and artificial molecules known generally by those who use technology related with nucleic acids, to thereby generate polymers that bind specifically to nucleic acids;

b) When used the term “nucleotide sequence” is the same as referring to a segment of a polymer containing subunits, in this case the nucleotides;

c) The term “target sequence” refers to a nucleotide sequence of Aspergillus fumigatus that is intended to be detected in the test, where the portion of nucleotides of the probe is designed to hybridize;

d) The term “PNA probe” refers to a polymer of subunits of PNA, which has a nucleotide sequence and is specific to hybridize with a target sequence of the microorganism of interest. PNA molecules are DNA mimics in which the negatively charged sugar-phosphate backbone structure is replaced by an achiral and electrically neutral formed by repeated N-(2-aminoethyl)glycine units;

e) When using the term “detectable fraction”, it refers to molecules that can be connected to the probe, to thereby render the probe detectable by an instrument or method;

f) The term “sample” refers to any biological sample that may contain the microorganism or target sequence for detection. Preferably the biological samples are in liquid form (example: blood, serum, bronchoalveolar lavage fluid and even sputum) or as tissue sample (example: biopsy).

### BRIEF DESCRIPTION OF THE DRAWINGS

FIG. 1 presents the partial alignment of the 28S rRNA sequences for probe selection. The complementary sequence of the FUM274 probe is shown above the alignment and the polymorphic positions are marked as well.

### DESCRIPTION

PNA Probe Design:

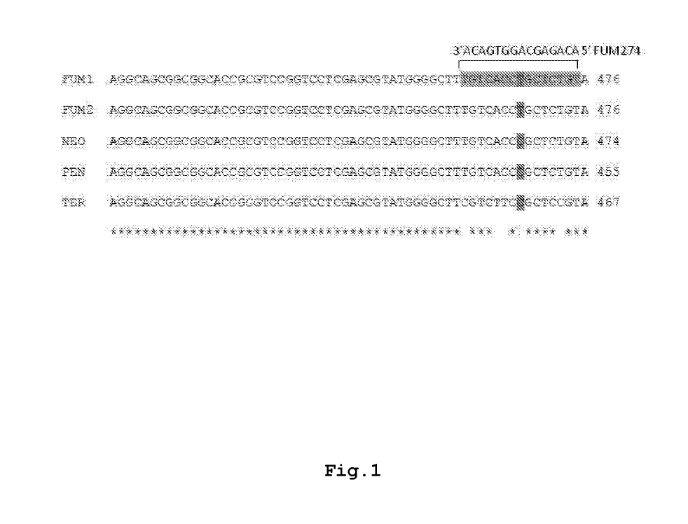

To identify an A. fumigatus oligonucleotide sequence to be used as probe, twenty four sequences of 28S rRNA available at the National Center for Biotechnology Information (NCBI) (http://www.ncbi.nlm.nih.gov) and SILVA (http://www.arb-silva.de/browser/) databases, were chosen. This selection contained eleven Aspergillus fumigatus sequences, six Penicillium sp., four Aspergillus terreus and 3 Neosartorya fischeri sequences. The regions of interest were selected using ClustalW (European Bioinformatics Institute; http://www.ebi.ac.uk/clustalw/) (examples of some of sequences were used in FIG. 1).

Other criteria were also considered important, such as guanines and cytokines percentage, secondary structures and hybridization temperature. The selected sequence, with the highest number of A. fumigatus sequences detected and the lowest number of non-A. fumigatus sequences detected, was 5′-ACA GAG CAG GTG ACA-3′. The sequence targeted the 28S rRNA between positions 274 and 288 of the A. fumigatus A1163 (Accession number ABDB01000088; SILVA database), and was therefore named FUM274. The probe lacked self-complementarity and presented 53% of guanines and cytokines.

Theoretical Evaluation of the PNA Probe Performance:

After the design of the probe, its performance was evaluated by determining the theoretical values for sensitivity and specificity. These parameters were evaluated with the software ProbeCheck available in the rRNA SILVA databases (http://www.arb-silva.de/fish-probes/probe-design/). Specificity was calculated as nAfs/(TnAf)×100, where nAfs stands for the number of non-Aspergillus fumigatus strains that did not react with the probe and TnAf is the total of non-Aspergillus fumigatus strains examined. Sensitivity was calculated as Afs/(TAfs)×100, where Afs stands for the number of Aspergillus fumigatus strains detected by the probe and TAfs is the total number of Aspergillus fumigatus strains present in the databases.

The search showed that FUM274 detected 79 out of 80 A. fumigatus 28S sequences available in the database that cover the alignment position of the selected probe and therefore the theoretical sensitivity was calculated as 98.8%. No other species presented sequences complementary to the probe and as such specificity reached 100%. Afterwards, the sequence was synthesized. The N terminus of the oligomer was connected to Alexa Fluor 594.

The PNA probe of this invention comprises preferably 15 nucleotides and may be at least 86% identical to the sequence SEQ ID No. 1-5′-ACA GAG CAG GTG ACA-3′, preferably 87%, 88%, 89%, 90%, 91%, 92%, 93%, 94%, 95%, 96%, 97%, 98%, 99%, 100% of similarity to SEQ ID No. 1-5′-ACA GAG CAG GTG ACA-3′.

Alternatively, this invention also contemplates variations of the nucleotide sequences of the probes. Such variations may include deletions, insertions, among others. For example on of the following sequences:

SEQ ID No. 2
5′- CTA CAG AGC AGG TGA -3′
SEQ ID No. 3
5′- CAG AGC AGG TGA CAA -3′
SEQ ID No. 4
5′- CTA CAG AGC AGG TGA CA -3′

Detectable Fraction of the PNA Probe:

Not limited to the following examples, the detectable fraction of PNA probe can include various types of molecules such as dextran conjugates, chromophores, fluorophores, radioisotopes, enzymes, haptens, chemiluminescent compound, among others.

As an example, among the fluorophores class those that are preferable for use are (but not limited to): Alexa series fluorophores, Alexa Fluor series, cyanines, 5- (and -6) Carboxy-2′,7′-dichlorofluorescein, 5-ROX (5-carboxy-X-rhodamine, triethylammonium salt.

Method:

The present invention presents a method for determining the presence of A. fumigatus using a nucleotide sequence with at least 86% of homology with the region of 15 nucleotides here described—SEQ ID No. 1.

The characteristics of the PNA probe used were previously described in this document with the referred sequences. The method can include the contact of a sample with PNA probe described herein with the fungus target sequence in appropriate hybridization conditions or appropriate in situ hybridization conditions. The method can be divided into: sample preparation (which may include a conidia germination step, when necessary), fixation, hybridization, wash and visualization of the results (see EXAMPLE 1). The method can be performed on adhered or suspended cells.

Hybridization Conditions

The following steps are a possible optimization of the hybridization conditions, without being a limitation of this invention:

There are several factors that impose and control the accuracy of the PNA probe hybridization to the target sequences. These include the percentage of formamide (or other denaturing chemical reagent), salt concentration and consequently the ionic strength, temperature of hybridization, the detergent concentration, pH and others. To determine the optimal hybridization conditions it may be necessary to fix the different factors and change each factor individually until a desirable discriminatory degree is achieved.

The closer a target sequence is from another non-target in the sample, the greater the stringency degree needed to define the various factors that influence the hybridization.

In this invention non-target sequences, can have only one different nucleotide in comparison to the target sequences, and as such an increased level of discrimination is necessary to avoid non-specific hybridizations.

PNA blocking probes can be used in this method to suppress these non-specific binding. To this purpose, it can be addressed these probes to non-target sequences similar to the target sequences. It is generally accepted that the blocking probes act forming thermodynamically more stable complexes than those formed between PNA probe and these same non-target sequences, avoiding the latter connection.

For the probes described in this document, the following conditions were detected:

Hybridization temperatures between 53° C. and 59° C. The strongest fluorescent signal was obtained at 55° C., for both hybridization in slides and hybridization in suspension.

Fixation step using ethanol concentrations ranging between 50% and 80%, but no differences were observed in the signal intensity.

The hybridization time was tested (30, 45, 60 e 90 min) but the times ranged between 45 minutes and 60 minutes were more efficient.

After the optimization of all the parameters referred above, the procedure that was found to result in a stronger fluorescent signal was as follows:

Smears of each fungal culture were prepared in appropriated slides for fluorescence microscopy observation.

Then the Smear Were:

Immersed in 4% (wt/vol) paraformaldehyde for 10 minutes, followed by 50% (vol/vol) ethanol, also for 10 minutes; The samples were air-dried and then covered with 20 μl of hybridization solution containing: 10% (wt/vol) dextran sulfate; mM NaCl; 30% (vol/vol) formamide; 0.1% (wt/vol) sodium pyrophosphate; 0.2% (wt/vol) polyvinylpirrolydone; 0.2% (wt/vol) Ficoll; 5 mM disodium EDTA; 0.1% (vol/vol) Triton X-100; 50 mM Tris-HCl (pH 7.5) and 200 nM of PNA probe; The samples were covered with coverslips placed in small wet boxes protected from light and incubated for 60 minutes at 55° C.; Afterwards, the coverslips were removed and the slides were submerged in a pre-warmed wash solution at 55° C. containing 5 mM Tris Base, 15 mM NaCl and 1% (vol/vol) Triton X-100 (pH 10). The washing step was also carried out for 30 minutes at 55° C. Subsequently, the slides were removed from the wash solution and dried at 55° C. in the same incubator for approximately 5 minutes. Before the microscope observation, a drop of non-fluorescent immersion oil was placed and covered with a coverslip. The slides were stored in the dark for a maximum of 24 hours before microscopy.

The hybridization can also be performed in suspension. In some cases this procedure helps almost totally eliminating the autofluorescence, namely the autofluorescence of the erythrocytes, in the case of blood samples. In this case the culture homogenized in sterile water is centrifuged (10,000×g for 5 minutes) and the pellet is homogenized in 500 μl 4% paraformaldehyde. After 1 hour, the cells centrifuged once again, in order to remove the paraformaldehyde, and the pellet is homogenized in 500 μl of 50% (vol/vol) ethanol. After 30 min of incubation at −20° C., the cells are homogenized once again in 100 μl of hybridization solution with 200 nM PNA probe and incubated at 55° C. for 1 hour. After hybridization, the cells were centrifuged and homogenized in 500 μl of wash solution (as described above) and incubated at 55° C. for 30 min. Finally, the cells are centrifuged to remove the wash solution and homogenized in 500 μl of sterile water. Next, 20 μl of the cell suspension are spread on microscope slides suitable for fluorescence or 200 μl are filtered through a membrane (pore size 0.2 μm, cellulose nitrate).

In order to check that the signal that was obtained was not related with autofluorescence, all samples were observed with the other filters available in the microscope. Further, a negative control was performed in each assay, following all the steps of the procedure but without the addition of probe to the hybridization solution.

Testing of the Probe Experimental Specificity and Sensitivity

Once the hybridization method was fully optimized, the experimental values of specificity and sensitivity of the PNA probe were tested. For this, the procedure was applied to eight A. fumigatus strains, twelve Aspergillus non-fumigatus (Aspergillus ibericus, Aspergillus ochraceus, Aspergillus versicolor, Aspergillus terreus, Aspergillus tubingensis, Aspergillus oryzae, 2 strains of Aspergillus flavus, 2 strains of Aspergillus Niger, Emericella nidulans var. echinulata and Neosartorya fisheri var. glabra), nine strains of filamentous fungus and yeasts (Penicillium brevicompactum, Penicillium chrysogenum, Mucor hiemalis, Trichoderma viride, Candida parapsilosis, Candida tropicalis, Candida glabrata and Candida albicans) and four bacterial strains that can be associated with pulmonary diseases (Pseudomonas aeruginosa PAO1, Pseudomonas aeruginosa CECT 111, Escherichia coli K12 and Staphylococcus aureus). The probe only hybridized with Aspergillus fumigatus strains. Therefore, in practical terms, specificity and sensitivity were 100% showing the good quality of the selected sequence regarding the capacity of discriminating A. fumigatus among other strains.

Aspergillus fumigatus Germination Assays:

Since it is known that filamentous fungus have different morphologic structures, such as hyphae or conidia, and since these last present a thick protective cellular structure, it was necessary to verify the probe performance in these different structures. A test using A. fumigatus conidia without a pre-germination step and overnight grown hyphae was performed. It was observed that an easily observable fluorescence signal was presented in hyphae.

Since it was important to minimize the time needed before carrying out the hybridization step, experiments were performed where the hybridization performance in different developmental states of A. fumigatus was monitored.

Conidia started to swell after only 2 hours, but this event was more evident after 4 hours. Moreover, in both times fluorescence signal was faint. Partial germination can be observed at 6 h and 8 h where apical growth of hyphae can be visualized. In this stage, fluorescence is much brighter, extending up to 12 h where full germination occurred. Because 6 h, was the time when the fluorescence signal-to-noise ratio started to be stronger, a germination step with this period of time, is probably sufficient for A. fumigatus detection.

Samples Preparation:

The samples to be analyzed can be obtained from blood, serum, sputum, bronchoalveolar lavage fluid, biopsies, water, among others.

In biopsies, the samples are cut in 3 to 5 mm slices and placed on slides. The hybridization step is performed directly in the biopsy.

The blood and sputum samples can be added to BACTEC™ Plus Aerobic/F (Becton Dickinson bottles) culture media, and incubated at 37° C., 120 rpm for at least 6 h allowing the conidia germination.

Some samples such as sputum may need a longer incubation period. The hybridization can be performed in slides or suspension.

Visualization of the Results:

This step can be performed in any epifluorescence microscope with a filter sensitive to fluorophore used. Other filters present in the microscope, which are not able to detect the fluorescent signal of the probe, were used to confirm the absence of autofluorescence.

Kit:

The present invention also refers to a kit that allows testing for the presence of fungi from the Aspergillus fumigatus genus.

The kit of the present invention comprises a PNA probe at least 86% identical to SEQ ID No. 1 and another reagents or compositions that are selected to perform the test.

The PNA probes to be used in the kit, its characteristics, and the method were previously referred in this document.

This invention can be used for both, analysis of the organism or analysis of nucleic acids extracted or derived from the organism of interest, implying that the source of the target sequence is not a limitation on this invention.

The following examples illustrate different steps for implementing the invention, without intending to limit any of them:

### EXAMPLE 1

### Distinction of Aspergillus fumigatus Strains from Other Filamentous Fungi

PNA Probe Sequence:

SEQ ID No. 1
5′- ACA GAG CAG GTG ACA -3′
(coupled to Alexa Fluor 594).

A. fumigatus and other filamentous fungi strains, capable to form conidia were maintained in Sabouraud dextrose agar or Potato dextrose agar for approximately 7 days at room temperature. For each experiment, conidia were harvested by flooding the agar surface with sterilized saline solution containing NaCl 8.00 g.L-1; KCl 0.2 g.L-1 ; Na2HPO4.2H2O 1.44 g.L-1; KH2PO4 0.24 g.L-1 (pH 7.4). Biomass was then suspended in the saline solution and collected with a pipette tip to a sterile tube. The heavier fragments were allowed to deposit in the bottom for 5-10 minutes and subsequently the supernatant was transferred to a new sterile tube.

The suspension is centrifuged (10 minutes; 10.000 g) to wash the sample. Subsequently 1×10⁶ cells ml⁻¹ of that suspension were resuspended in peptone-yeast extract-glucose (PYG) containing peptone 1 g.L⁻¹; yeast extract 1 g.L⁻¹ and glucose 3 g.L⁻¹ (pH 5) and placed overnight (approximately 16 hours) at 37° C., 120 rpm, allowing full conidia germination. At the end, suspensions were centrifuged for 10 minutes, 10.000 g, being the supernatant replaced by saline solution. This last step was repeated two times to remove any residue of the growing media. The suspensions were then dispensed in fluorescence microscopy slides and allowed to dry at an incubator at 55° C. (5 mins) or left to air dry.

Fixation:

For preventing the loss of 28S rRNA during the hybridization process, the samples were immersed in a solution of 4% paraformaldehyde (wt/vol) and 50% ethanol (vol/vol) for 10 minutes each.

Hybridization:

After fixation, samples were then covered with a drop of hybridization solution containing: 10% (wt/vol) dextran sulfate; 10 mM NaCl; 30% (vol/vol) formamide; 0.1% (wt/vol) sodium pyrophosphate; 0.2% (wt/vol) polyvinylpirrolidone; 0.2% (wt/vol) Ficol; 5 mM disodium EDTA; 0.1% (vol/vol) Triton X-100; 50 mM Tris-HCl (pH 7.5); and 200 nM PNA probe. The samples were covered with coverslips (to assure an homogeneous spreading of the probe) placed in small wet boxes (to prevent the evaporation of the hybridization solution) protected from light and incubated for 60 minutes at 55° C.

Wash:

Subsequently, the coverslips were removed and the slides were immersed in a wash solution pre-warmed at 55° C. containing 5 mM Tris Base, 15 mM NaCl and 1% (vol/vol) Triton X-100 (pH 10). The washing step takes 30 minutes long, at 55° C.

Subsequently, the slides were removed from the wash solution and dried at 55° C., in the same incubator, for approximately 5 minutes. Before the microscope visualization, a drop of non-fluorescent immersion oil was placed and covered with a coverslip. The slides were kept in the dark for a maximum period of 24 hours before microscopy.

Results:

The results were obtained through the observation in a fluorescence microscope with a filter capable of detecting the fluorochrome Alexa Fluor 594 bonded to the PNA probe.

### EXAMPLE 2

### Detection of A. fumigatus in Different Clinical Samples (Blood and Sputum)

PNA Probe Sequence:

SEQ ID No. 1
5′- ACA GAG CAG GTG ACA -3′
(coupled to Alexa Fluor 594).

Sample Preparation:

Ten ml of defibrinated sheep blood or 1 ml of artificial sputum media were added to BACTEC™ Plus Aerobic/F Medium and incubated at 37° C., 120 rpm. After 6 hours germination (minimal germination time required to perform brighter fluorescence signal), 1 ml was recovered from each culture to perform hybridization on glass slides.

Fixation:

The fixation was performed according to the procedure described in Example 1.

Hybridization:

The hybridization was performed according to the described in Example 1 with the slightly difference of using distilled water, instead of saline solution, in the hybridization process steps, with the purpose of better disrupting cells (example: blood).

Wash:

The washing step was performed according to the procedure described in Example 1.

Results:

The results were obtained through the observation in a fluorescence microscope with a filter capable of detecting the fluorochrome Alexa Fluor 594 bonded to the PNA probe. Lisbon, Jun. 19, 2014.

### REFERENCES

Lomakin, A.; Frank-Kamenetskii, M. D. A theoretical analysis of specificity of nucleic acid interactions with oligonucleotides and peptide nucleic acids (PNAs). J. Mol. Biol. 1998, 276, 57-70.

Perry-O'Keefe H, Rigby S, Oliveira K, Sorensen D, Stender H, Coull J, Hyldig-Nielsen J J: Identification of indicator microorganisms using a standardized PNA FISH method. J Microbiol Methods 2001, 47(3):281-292.

Cerqueira L, Fernandes R M, Ferreira R M, Carneiro F, Dinis-Ribeiro M, Figueiredo C, Keevil C W, Azevedo N F, Vieira M J: PNA-FISH as a new diagnostic method for the determination of clarithromycin resistance of Helicobacter pylori. BMC Microbiol 2011, 11(1):101.

Almeida C, Azevedo N F, Fernandes R M, Keevil C W, Vieira M J: Fluorescence in situ hybridization method using a peptide nucleic acid probe for identification of Salmonella spp. in a broad spectrum of samples. Appl Environ Microbiol 2010, 76(13):4476-4485

Oliveira K, Haase G, Kurtzman C, Hyldig-Nielsen J J, Stender H: Differentiation of Candida albicans and Candida dubliniensis by fluorescent in situ hybridization with peptide nucleic acid probes. J Clin Microbiol 2001, 39(11):4138-4141.

Teertstra W R, Lugones L G, Wosten H A: In situ hybridisation in filamentous fungi using peptide nucleic acid probes. Fungal Genet Biol 2004, 41(12):1099-1103.

Shinozaki M, Okubo Y, Sasai D, Nakayama H, Murayama S Y, Ide T, Wakayama M, Hiruta N, Shibuya K: Identification of Fusarium species in formalin-fixed and paraffin-embedded sections by in situ hybridization using peptide nucleic acid probes. J Clin Microbiol 2010, 49(3):808-813

U.S. Pat. No. 5,539,082—Peptide Nucleic Acids

US2005053944/WO9712995/EP1477572—Methods and kit for hybridization analysis using peptide nucleic acid probes.

1. A PNA probe for the detection and/or quantification of Aspergillus fumigatus characterized in that it comprises at least one sequence with at least 86% similarity to SEQ ID No. 1-5′-ACA GAG CAG GTG ACA-3′.

2. The PNA probe, according to claim 1, characterized in that it comprises at least one of the following sequences:
SEQ ID No. 1
5′- ACA GAG CAG GTG ACA -3′;
SEQ ID No. 2
5′- CTA CAG AGC AGG TGA -3′
SEQ ID No. 3
5′- CAG AGC AGG TGA CAA -3′
SEQ ID No. 4
5′- CTA CAG AGC AGG TGA CA -3′

3. The PNA probe, according to claim 1 characterized in that it capable of detecting the target sequences in rRNA, rDNA or the sequences complementary to A. fumigatus rRNA.

4. The PNA probe, according to claim 1, characterized in that it additionally comprises one sequence with at least 86% similarity to SEQ ID No. 1-5′-ACA GAG CAG GTG ACA-3′.

5. The PNA probe, according to claim 1, characterized in that it is connected to at least one type of detectable fraction.

6. The PNA probe, according to claim 5, characterized in that the type of detectable fraction of the probe is selected from one of the following groups: a conjugate, a branched detection system, a chromophore, a fluorophore, radioisotope, an enzyme, a hapten or a luminescent compound.

7. The PNA probe, according to claim 6, characterized in that the fluorophore group is at least one of the following: fluorophores of the Alexa series, Alexa Fluor series, cyanines, 5-(and -6) Carboxy-2′,7′-dichlorofluorescein, the 5-ROX (5-carboxy-X-rhodamine, triethylammonium salt).

8. A kit for detecting A. fumigatus, characterized in that it comprises at least one of the probes described in claim 1.

9. The kit, according to claim 8, characterized in that it further comprises at least one of the following solutions: one fixation solution, one hybridization solution and one washing solution.

10. The kit, according to claim 9, characterized in that the fixation solution comprises paraformaldehyde and ethanol, namely 2-8% (wt/vol) of paraformaldehyde and 25-90% (vol/vol) of ethanol.

11. The kit, according to claim 9, characterized in that the hybridization solution comprises formamide.

12. A method for detection of A. fumigatus, characterized in that it uses the PNA probes described in claim 1 and it comprises the following steps:
a. PNA probe contact with the biological samples, previously removed from the human body;
b. PNA probe hybridization with the target sequence of the microorganisms present in the referred samples;
c. Hybridization detection as indication of the referred detection and quantification of the referred samples.

13. The method, according to claim 12, characterized in that the biological sample is derived from blood, sputum, bronchoalveolar lavage fluid, biopsies, air, food or water.

14. The method, according to claim 12, characterized in that the hybridization occurs by fluorescence.

15. Use of PNA probes, as described in claim 1, characterized in that it is applied in a methodology for detecting A. fumigatus in biological samples.

16. Use of the kit, as described in claim 8, characterized in that it is applied in the detection of A. fumigatus in biological samples.


SEQUENCE LISTING (8 sequences)

SEQ ID NO: 1 - DNA, 15 bp, Aspergillus fumigatus
acagagcaggtgaca

SEQ ID NO: 2 - DNA, 15 bp, Aspergillus fumigatus
ctacagagcaggtga

SEQ ID NO: 3 - DNA, 15 bp, Aspergillus fumigatus
cagagcaggtgacaa

SEQ ID NO: 4 - DNA, 17 bp, Aspergillus fumigatus
ctacagagcaggtgaca

SEQ ID NO: 5 - DNA, 60 bp, Aspergillus fumigatus
aggcagcggcggcaccgcgtccggtcctcgagcgtatggggctttgtcacctgctctgta

SEQ ID NO: 6 - DNA, 60 bp, Neosartorya fischeri
aggcagcggcggcaccgcgtccggtcctcgagcgtatggggctttgtcacccgctctgta

SEQ ID NO: 7 - DNA, 60 bp, Aspergillus terreus
aggcagcggcggcaccgcgtccggtcctcgagcgtatggggcttcgtcttccgctccgta

SEQ ID NO: 8 - DNA, 15 bp, Aspergillus fumigatus
acagtggacgagaca